(19) United States
(12) Patent Application Publication
(10) Pub. No.: US 2014/0076333 A1
(43) Pub. Date: Mar. 20, 2014

(54) Sleep Apnea Dental Appliance and Method of Constructing

(71) Applicants: Suzzette L. Ona, Kentwood, MI (US); Robert Kennicott, Kentwood, MI (US)

(72) Inventors: Suzzette L. Ona, Kentwood, MI (US); Robert Kennicott, Kentwood, MI (US)

(21) Appl. No.: 14/028,821

(22) Filed: Sep. 17, 2013

Related U.S. Application Data
(60) Provisional application No. 61/703,333, filed on Sep. 20, 2012.

(51) Int. Cl. A61F 5/56; A61C 19/05; A61C 9/00

(52) U.S. Cl. CPC A61F 5/566; A61C 9/0006; A61C 19/05
USPC 128/848; 264/16

(57) ABSTRACT

A sleep apnea dental appliance and a method for constructing said appliance. The sleep apnea dental appliance and the method for constructing said dental appliance offers patients a mandibular advancement splint that is custom fitted to their upper and lower teeth. The apparatus reduces strain to the patient's mandible while in the advanced position. The sleep apnea dental appliance utilizes a maxillary dental arch and a mandibular dental arch fused together in a particular configuration determined by precise measurements taken by dental professional. The method of construction allows the apparatus to be constructed at relatively low cost which can translate into low costs for the patient.

The current application claims a priority to the U.S. Provisional Patent application Ser. No. 61/703,333 filed on Sep. 20, 2012.

### FIELD OF THE INVENTION

The present invention relates generally to an oral appliance, more specifically, to a dental appliance and method of constructing said dental appliance for the treatment of obstructive sleep apnea.

### BACKGROUND OF THE INVENTION

Sleep apnea is a disorder that occurs when an individual sufferers from lengthy abnormal pauses in breathing during their normal sleep cycle. These abnormal pauses result in daytime sleepiness and fatigue normally associated with a significant level of sleep disturbance. In the most common form of sleep apnea, known as obstructive sleep apnea (OSA), breathing is frequently interrupted by a physical block to airflow despite respiratory efforts during sleep. Typically, OSA patients suffer from structural features that give rise to a narrowed airway which are exacerbated by low muscle tone and excessive soft tissue around the airway. The most common symptoms observed by individual's suffering from sleep apnea are fatigue and daytime sleepiness usually at levels associated with significant sleep disturbance. For those observing an OSA patient the symptoms are characterized by excessive and loud snoring with frequent sudden pauses. It is believed that sleep apnea affects one in every fifteen adults in the United States, but due to the plurality of factors that could potentially results in OSAs most common symptoms, current figures could be a very conservative estimate. While sleep apnea is commonly associated with sleep disturbance symptoms, it has also been linked with an increased risk of cardiovascular disease, stroke, high blood pressure, arrhythmias, diabetes, and sleep deprived driving accidents.

Due to the known risks associated with OSA, various solutions have been created in an attempt to resolve or treat the symptoms associated with OSA. Some of these treatment options include such as positive airway pressure devices and mandibular advancement splints. Positive airway pressure devices require the patient to wear a mask that covers their nose, mouth, or both airway openings in order to provide positive air pressure during the inhalation forcing air into the patient's lungs reducing the symptoms of obstructive sleep apnea. These devices include continuous positive airway pressure (CPAP), variable positive airway pressure (VPAP), and automatic positive airway pressure (APAP) machines. Typically positive airway pressure mandibular advancement splints are prescribed to individuals suffering from severe cases of OSA. Mandibular advancement splints are appliances that are worn by a patient suffering from mild to moderate sleep apnea. Mandibular advancement splints are worn by a patient similar to a mouth guard and are designed to move the patient's lower jaw into forward position relative to their upper jaw moving the patient's jaw away from their airway opening and tightening some of the soft neck tissue found around the patient's neck. Although both options are able to treat some cases of sleep apnea, most suffer from several disadvantages that makes their implementation cumbersome.

Positive airway pressure devices are cumbersome to use and require the patient to wear a mask over their nose or mouth while they sleep. The masks can be uncomfortable for some patients. Additionally these masks require a connection to a central unit that generates the positive airway pressure. The connection to the central unit is accomplished with a hose. In some situations this hose can become entangled or compressed restricting the positive airway pressure required by the patient. Another disadvantage associated with the positive airway pressure devices is that they require an electrical connection or a battery backup in order to operate. In many ways mandibular advancement splints address the short comings associated with the positive airway pressure devices but suffer from their own unique disadvantages. Many of the disadvantages associated with mandibular advancement splits are a result of the size, complexity, and construction. Some of the mandibular advancement splints allow the patient to adjust the forward positioning of their lower jaw. These devices utilize complex mechanical components in order to allow this adjustment limiting the device to patients with mouth large enough to comfortably wear the devices. Another disadvantage seen in both mandibular advancement splints and positive airway pressure devices is the cost to the patient.

It is therefore the object of the present invention to provide sleep apnea dental appliance and a method for constructing said appliance. The sleep apnea dental appliance and the method for constructing said dental appliance offers patients a mandibular advancement splint that is custom fitted to their upper and lower teeth. The apparatus reduces strain to the patient's mandible while in the advanced position. The sleep apnea dental appliance utilizes a maxillary dental arch and a mandibular dental arch fused together in a particular configuration determined by precise measurements taken by dental professional. The method of construction allows the apparatus to be constructed at relatively low cost which can translate into low costs for the patient.

### BRIEF DESCRIPTIONS OF THE DRAWINGS

FIG. 1 is a perspective view displaying the sleep apnea dental appliance as per the current embodiment of the present invention.

FIG. 2 is a frontal view displaying the maxillary dental arch of the sleep apnea dental appliance as per the current embodiment of the present invention.

FIG. 3 is a frontal view displaying the mandibular dental arch of the sleep apnea dental appliance as per the current embodiment of the present invention.

FIG. 4 is a right side view displaying the sleep apnea dental appliance as per the current embodiment of the present invention.

FIG. 5 is a left side view displaying the sleep apnea dental appliance as per the current embodiment of the present invention.

FIG. 6 is a frontal view displaying the sleep apnea dental appliance as per the current embodiment of the present invention.

FIG. 7 is a top elevational view displaying the inner portion of the maxillary dental arch as per the current embodiment of the present invention.

FIG. 8 is a bottom elevational view displaying the inner portion of the mandibular dental arch as per the current embodiment of the present invention.

FIG. 9 is a rear view displaying the sleep apnea dental appliance as per the current embodiment of the present invention.

### DETAIL DESCRIPTIONS OF THE INVENTION

All illustrations of the drawings are for the purpose of describing selected versions of the present invention and are not intended to limit the scope of the present invention.

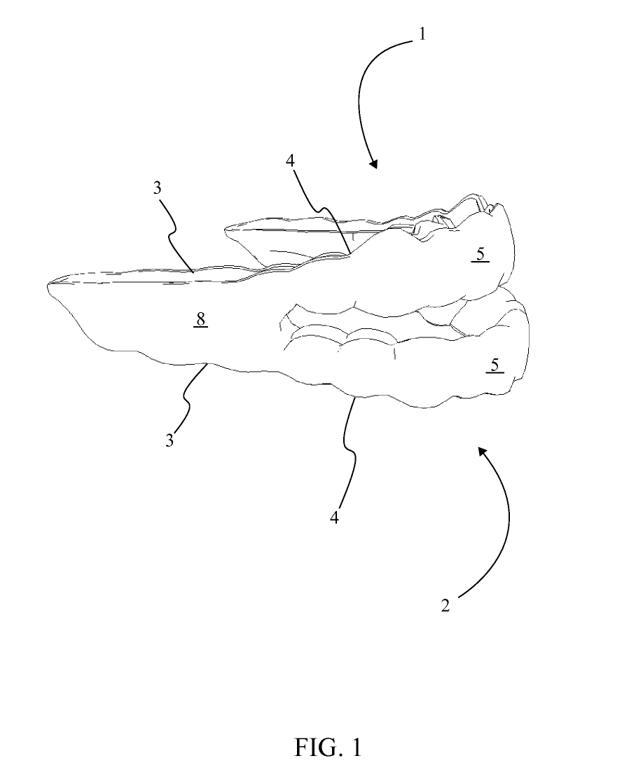

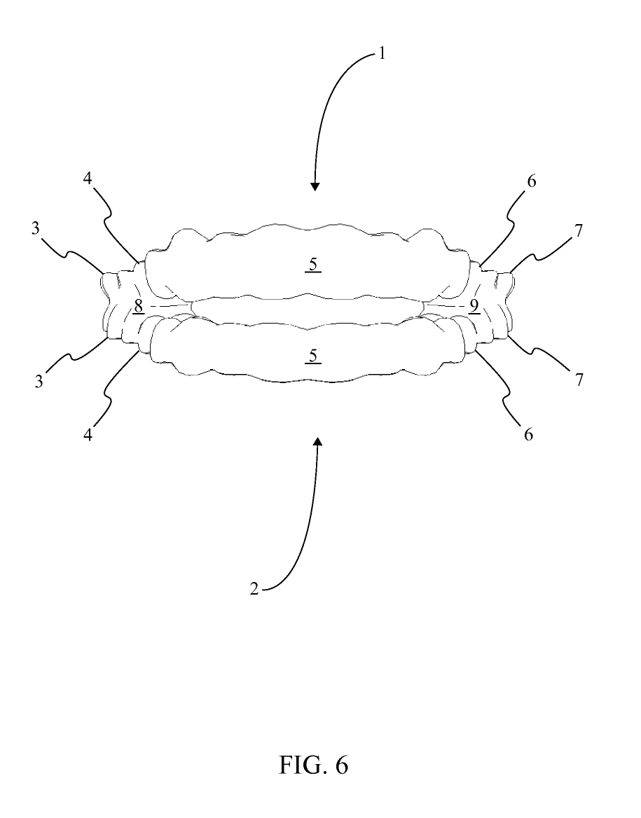

Referencing FIG. 1 and FIG. 6, a sleep apnea dental appliance is an orally mounted device that advances a patient's lower jaw (mandible) to a forward position treating obstructive sleep apnea. The sleep apnea dental appliance accomplishes this by repositioning the patient's mandible forward causing the patient's soft neck tissue to move away from the patient's esophagus, reducing obstruction to the patient's airway which may result in obstructive sleep apnea in a supine position. The sleep apnea dental appliance comprises a mandibular dental arch 2, a maxillary dental arch 1, a first advancement structure 8, and a second advancement structure 9. The mandibular dental arch 2 is specifically formed to engage the patient's lower teeth permitting the sleep apnea dental appliance advance the patient's lower jaw. The maxillary dental arch 1 is provided as a complementary component to the mandibular dental arch 2 that engages to the patient's upper teeth. Both the first advancement structure 8 and the second advancement structure 9 are provided as structural elements that joins the maxillary dental arch 1 and the mandibular dental arch 2 in a particular arrangement.

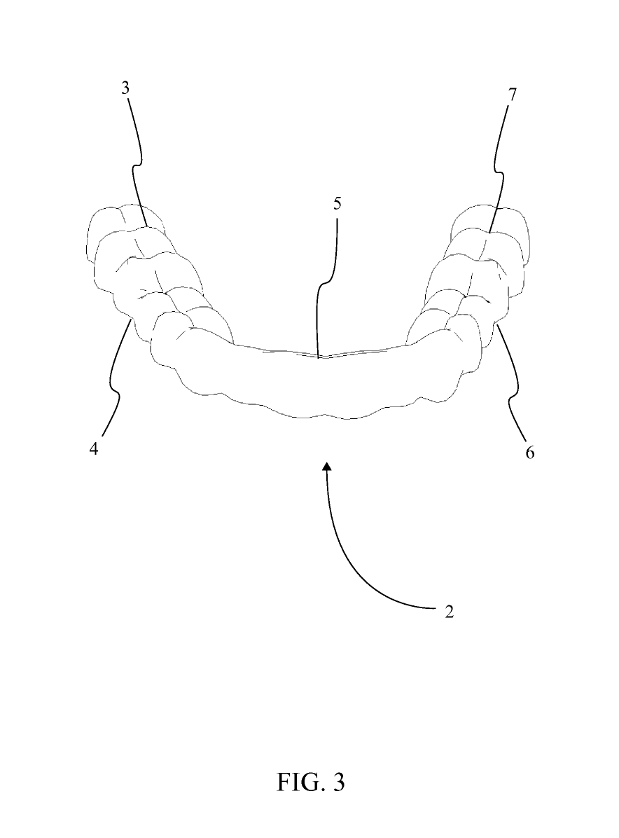

Referencing FIG. 3 and FIG. 6, the mandibular dental arch 2 is the portion of the sleep apnea dental appliance that engages the patient's lower teeth. The mandibular dental arch 2 is created from an impression of the patient's lower teeth providing a particular shape and form that surrounds each of the patient's lower teeth. The mandibular dental arch 2 is fused to the first advancement structure 8 and the second advancement structure 9. Through the particular arrangement provided by the first advancement structure 8 and second advancement structure 9 with the maxillary dental arch 1, the mandibular dental arch 2 is positioned in front of the maxillary dental arch 1. The positioning of the mandibular dental arch 2 advances the patient's lower jaw tightening soft neck tissue surrounding the patient's esophagus reducing the likelihood of constriction/collapse of the patient's upper airway while in the supine position.

Referencing FIG. 3 and FIG. 6, the mandibular dental arch 2 provided in the current embodiment of the present invention, comprises a first molar region 3, a first premolar region 4, a cuspid/incisor region 5, a second premolar region 6, and a second molar region 7. The first molar region 3, the first premolar region 4, the cuspid/incisor region 5, the second premolar region 6, and the second molar region 7 of the mandibular dental arch 2, are found arcuately positioned in a manner that corresponds to the distribution of the patient's lower teeth. It should be noted that due to the bilateral symmetry of a patient's lower teeth, the first molar region 3 and the first premolar region 4, as well as the second molar region 7, and the second premolar region 6 of the mandibular dental arch 2 are non-specifically associated with either the patient's right molars and premolars or the left molars and premolars. The first premolar region 4 is found arcuately positioned between the first molar region 3 and the cuspid/incisor region 5, wherein the arcuate positioning of the first premolar region 4, the cuspid/incisor region 5, and the first molar region 3 coincide with the arc-like distribution of the patient's corresponding lower teeth. The cuspid/incisor region 5 is found arcuately positioned between the first premolar region 4 and the second premolar region 6. The second premolar region 6 is found arcuately positioned between the cuspid/incisor region 5 and the second molar region 7.

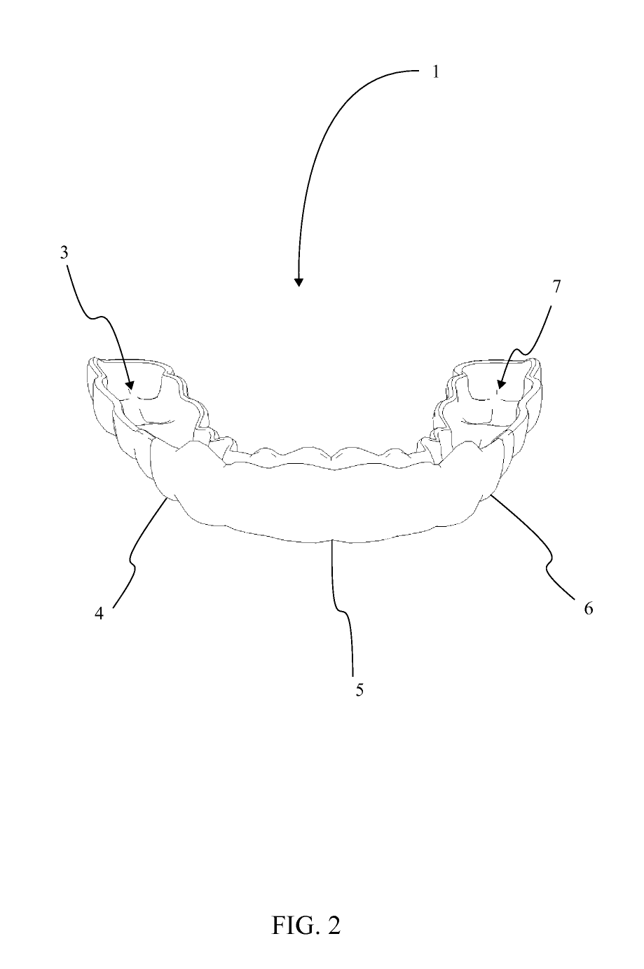

Referencing FIG. 2 and FIG. 6, the maxillary dental arch 1 is provided as the portion of the sleep apnea dental appliance that engages the patient's upper teeth. The maxillary dental arch 1 is found positioned parallel to the mandibular dental arch 2, wherein the maxillary dental arch 1 and the mandibular dental arch 2 are found parallel, at least in part, through the relationship between the patient's upper teeth and the patient's lower teeth. The maxillary dental arch 1 is formed from impressions of the patient's upper teeth. The maxillary dental arch 1 is provided with a particular shape allowing it to surround each of the patient's upper teeth. The maxillary dental arch 1 is fused to the first advancement structure 8 and the second advancement structure 9 opposite the positioning of the mandibular dental arch 2. The maxillary dental arch 1 functions cooperatively with the mandibular dental arch 2 and serves as an anchor point for maintaining the mandibular dental arch 2 in the forward position.

Referencing FIG. 2 and FIG. 6, the maxillary dental arch 1 provided in the current embodiment of the present invention, comprises a first molar region 3, a first premolar region 4, a cuspid/incisor region 5, a second premolar region 6, and a second molar region 7. The first molar region 3, the first premolar region 4, the cuspid/incisor region 5, the second premolar region 6, and the second molar region 7 of the maxillary dental arch 1, are found arcuately positioned in a manner that corresponds to the distribution of the patient's upper teeth. It should be noted that due to the bilateral symmetry of a patient's upper teeth, the first molar region 3 and the first premolar region 4, as well as the second molar region 7, and the second premolar region 6 of the mandibular dental arch 2 are non-specifically associated with either the patient's right molars and premolars or the left molars and premolars. The first premolar region 4 is found arcuately positioned between the first molar region 3 and the cuspid/incisor region 5, wherein the arcuate positioning of the first premolar region 4, the cuspid/incisor region 5, and the first molar region 3 coincide with the arc-like distribution of the patient's corresponding upper teeth. The cuspid/incisor region 5 is found arcuately positioned between the first premolar region 4 and the second premolar region 6. The second premolar region 6 is found arcuately positioned between the cuspid/incisor region 5 and the second molar region 7.

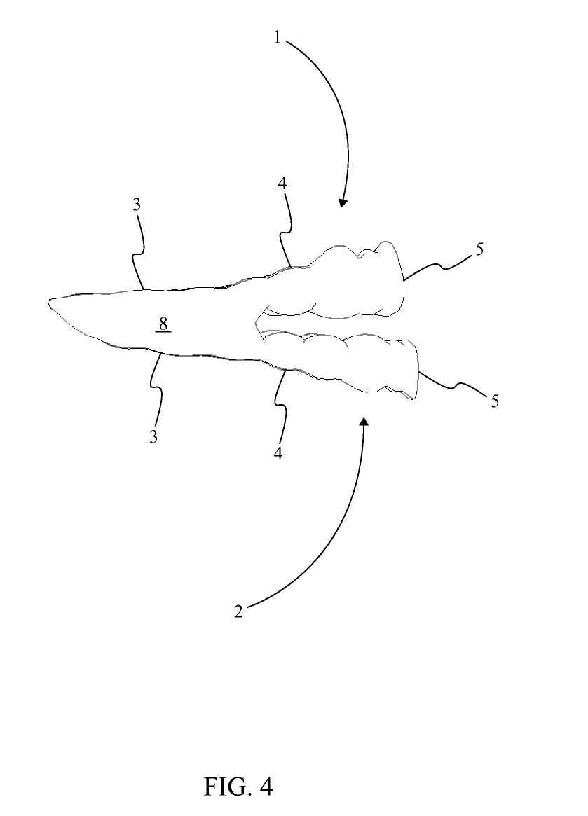

Referencing FIG. 4-6, the first advancement structure 8 and the second advancement structure 9 are structural elements that join the mandibular dental arch 2 and the maxillary dental arch 1 in the particular arrangement resulting in the advancement of the patient's lower jaw. The first advancement structure 8 and the second advancement structure 9 are distributed on the sleep apnea dental appliance according with bilateral symmetry, resulting in the first advancement structure 8 being positioned opposite the second advancement structure 9 across a plane that bisects through the cuspid/incisor region 5 of both the maxillary dental arch 1 and the mandibular dental arch 2. The first advancement structure 8 is found fused, at least in part, to the first molar region 3 of the maxillary dental arch 1 and the first premolar region 4 of the maxillary dental arch 1. The first advancement structure 8 is found fused, at least in part, to the first molar region 3 of the mandibular dental arch 2 and to the first premolar region 4 of the mandibular dental arch 2, opposite the maxillary dental arch 1. The second advancement structure 9 is found fused, at least in part, to the second molar region 7 of the maxillary dental arch 1, and to the second premolar region 6 of the maxillary dental arch 1. The second advancement structure 9 is found fused, at least in part, to the second molar region 7 of the mandibular dental arch 2, and to the second premolar region 6 of the mandibular dental arch 2, opposite the maxillary dental arch 1. Both the first advancement structure 8 and the second advancement structure 9 are described to be fused in at least in part, it should be noted that fusing the mandibular dental arch 2 and the maxillary dental arch 1 to both the first advancement structure 8 and the second advancement structure 9 is greatly dependent on the variations in size and shape of a patient's jaw. The sleep apnea dental appliance is particularly configured to fit to a patient's jaw resulting in variations in the construction and positioning of the components of the sleep apnea dental appliance.

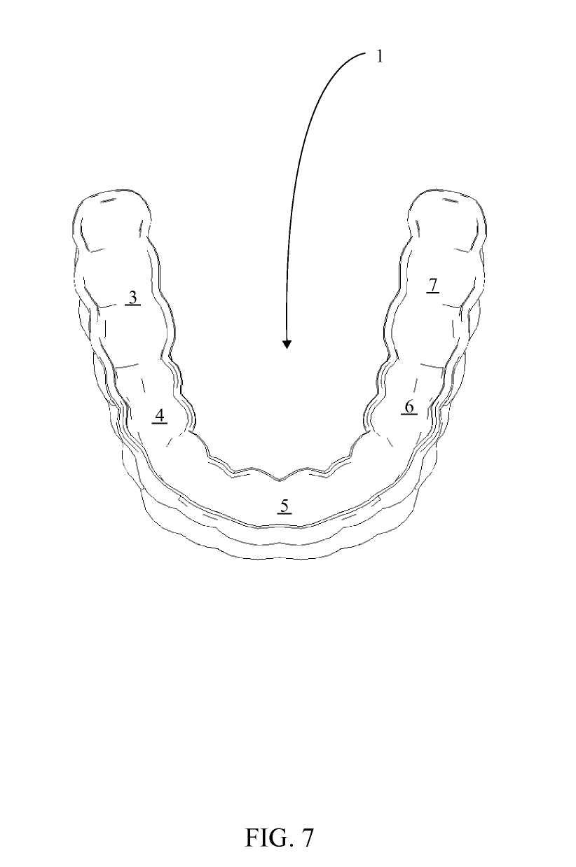

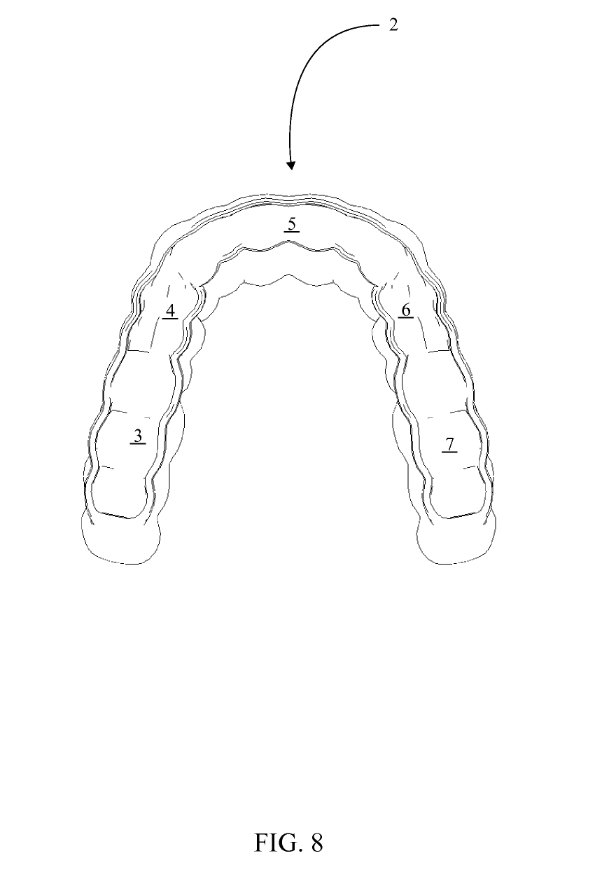

Referencing FIG. 6-9, in the current embodiment of the present invention, the mandibular dental arch 2, the maxillary dental arch 1, the first advancement structure 8, and the second advancement structure 9 are constructed of a pliable material. The use of a pliable material provides the sleep apnea dental appliance with a material that is more comfortable to wear for extended periods of time while additionally permitting slight movement of the patient's jaw preventing long term discomfort. The pliable material that is utilized by the present invention may include but is not limited to Ethylene Vinyl Acetate (EVA) as well as a variety of other polymers and copolymers utilized in the construction of dental appliances. It should be noted that the maxillary dental arch 1 and the mandibular dental arch 2 are constructed of a pliable material but that the first advancement structure 8 and the second advancement structure 9 may be constructed of a different pliable material due to the function of the both the first advancement structure 8 and the second advancement structure 9. In the current embodiment of present invention the first advancement structure 8 and the second advancement structure 9 may be constructed of pliable materials that include but are not limited to Lang Clear Cold Cure Acrylic as well as other combinations of synthetic resins.

Referencing FIG. 6-9, in the current embodiment of the present invention, the mandibular dental arch 2 and the maxillary dental arch 1 are provided with a thickness ranging between 0.04 mm and 0.10 mm in thickness. The thickness of both the mandibular dental arch 2 and the maxillary dental arch 1 related are dependent the dentist that is fabricating the sleep apnea dental appliance to diagnose the patient as being a bruxer. The dentist needs to determine if the patient is a severe bruxer, moderate bruxer, or mild to no bruxer prior to construct the mandibular dental arch 2 and the maxillary dental arch 1 in order to ensure the durability of the dental appliance while the patient is asleep. Dependent on the determination, the particular thicknesses are selected for a severe bruxer, medium bruxer, and a mild to no bruxer, wherein the thicknesses of the appliance for a sever bruxer is 0.08 mm, 0.06 mm for medium bruxer, and 0.04 mm for mild to no bruxer patient.

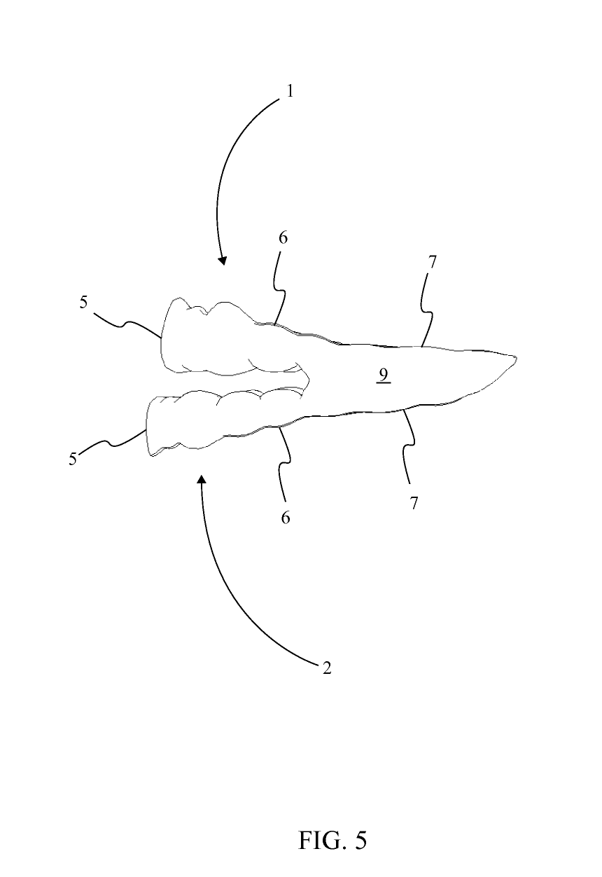

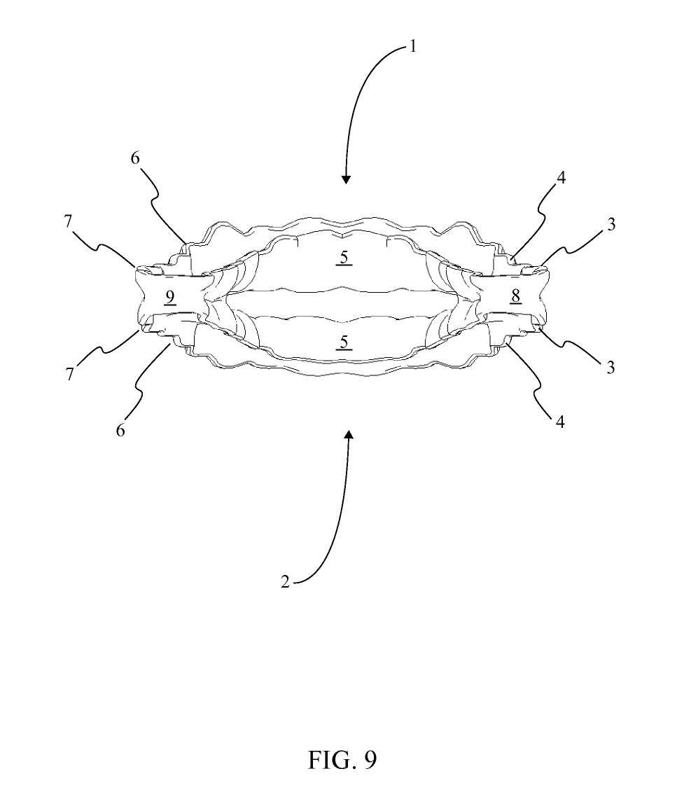

Referencing FIG. 4, FIG. 5, and FIG. 9, in the current embodiment of the present invention, the first advancement structure 8 and the second advancement structure 9 are each constructed with a thickness of 3.0 mm. The first advancement structure 8 and the second advancement structure 9 separate the maxillary dental arch 1 from the mandibular dental arch 2 but additionally provide enough resistance to prevent a patients from being able to close their mouths. The 3.0 mm thickness of the first advancement structure 8 and the second advancement structure provides the sufficient support preventing a patient from repositioning their jaw or closing their mouth while additionally being appropriately sized to comfortably fit within the mouth of an adult patient.

Referencing FIG. 4 and FIG. 5, in the current embodiment of the present invention, the cuspid/incisor region 5 of the mandibular dental arch 2 is found horizontally offset by 3.0 mm to 5.0 mm from the cuspid/incisor region 5 of the maxillary dental arch 1. The offset positioning of the cuspid/incisor region 5 of the mandibular dental arch 2 to the cuspid/incisor region 5 of the maxillary dental arch 1 is provided specifically as the means of advancing the patient's lower jaw forward and thus relieving compression to the esophagus caused by soft neck tissue. The offset distance of 3.0 mm to 5.0 mm is provided as the estimated range determined to comfortably fit most patients. The offset distance is measured for a particular patient through the use of a bite gauge measurement device. The bite gauge measurement device determines a comfortable offset distance for the particular patient to protrude their lower jaw. It should be noted that while the range for the offset distance is described as being between 3.0 mm and 5.00 mm from the cuspid/incisor region 5 of the mandibular dental arch 2 and the cuspid/incisor region 5 of the maxillary dental arch 1, that the actual distances may include measurements beyond the ones described based on known variation between patient's.

A method of constructing the sleep apnea dental appliance describes a low cost alternative process that is specific to construction of the sleep apnea dental appliance. The method of constructing the sleep apnea dental appliance comprises the steps of receiving a patient diagnosed with obstructive sleep apnea; determining the patient qualified for the sleep apnea dental appliance; creating a set of impression from the patient's upper teeth and the patient's lower teeth; measuring the bite relationship between the patient's upper teeth and the patient's lower teeth; creating a pair of stone models from the set of impressions; creating a pair of dental arches from the pair of stone models; and fusing the pair of dental arches in order to form the sleep apnea dental appliance.

The patient is diagnosed with obstructive sleep apnea by a physician where upon the physician would refer the patient to dental professional qualified to perform the necessary steps required to construct the sleep apnea dental appliance. The qualified dental profession would receive the patient and further perform their own diagnosis in order to determine if the patient is a candidate for the sleep apnea dental appliance. The qualified dental profession may ensure that the any preexisting dental disease is treated prior to prescribing the use of the sleep apnea dental appliance in order to ensure no complications as a result of inadequate teeth foundation. Such pre-existing dental conditions that must be treated prior to prescribing the sleep apnea dental appliance include but are not limited to gum disease, and tooth decay, as well a plurality of additional conditions that can result in tooth eruption and tooth rotation. It should be noted that patients with existing birth teeth, fixed crowns and bridge, partial or removable dentures, and full dentures over implants would not be disqualified from being prescribed the sleep apnea dental appliance since the aforementioned conditions would provided adequate foundations for the maxillary dental arch 1 and the mandibular dental arch 2. Additionally, it should be noted that the qualified dental professional would be able to diagnose the patient as being a bruxer. If the patient is diagnosed as being a bruxer, the qualified dental professional would be able to determine an appropriate thickness for the mandibular dental arch 2 and the maxillary dental arch 1 that would ensure the durability of the sleep apnea appliance for the patient.

The qualified dental professional would then create a set of impression from the patient's upper teeth and the patient's lower teeth. The dental professional would create a set of impressions from the patient's upper teeth and the patient's lower teeth through the use of a molding putty and tray. The molding putty is a compound that when activated will begin to harden conforming to the shape of adjacent structures and solid bodies. The tray is a container that retains the molding putty during to prevent it from deforming while it is still hardening. The qualified dental professional would activate the molding putty through the specific means required by the particular molding putty utilized. The molding putty may be activated by heating to a specific temperature or by combining it with another compound initiating a chemical reaction that solidifies both compounds. It should be noted that the qualified dental professional shall herein after be understood as the same entity. The dental professional would place an amount of the activated molding putty within a first tray and a second tray. The amount of activated molding putty should be sufficient to adequately surround the patient's upper teeth and the patient's lower teeth. The dental professional would align the molding putty in the first tray with the patient's upper teeth. The dental professional would then press the molding putty of the first tray in the patient's upper teeth, surrounding the upper teeth in the molding putty forming an impression. With the first tray still in position the dental professional would then align the molding putty of the second tray with the patient's lower teeth. The dental professional would then press the molding putty of the second tray into the patient's lower teeth, surrounding the lower teething in the molding putty forming an impression. The first tray and the second tray would be removed from their positioning with the patient's upper teeth and the patient's lower teeth, respectively, after a particular amount of time has passed. The particular amount of time is dependent on time it takes for the molding putty of the first tray and the molding putty of the second tray to sufficiently harden forming an impression of the patient's upper teeth and an impression of the patient's lower teeth.

In the current embodiment of the present invention, the molding putty and tray utilized in the creation of the pair of impressions utilizes tray putty system similar to the easy tray custom impression trays or a Kerr extrude XP putty with nu radiance warm and form mouth trays. In one of these tray systems warm water would be used to activate the putty which could then molded around the patient's upper teeth and lower teeth forming an impression for each. Another try system would combine a first putty compound and a second putty compound in equal parts forming the activated molding putty. The activated molding putty would then be separated into a first tray and a second tray which would then be aligned with the patient's upper teeth and the patient's lower teeth and in order to form the impressions.

The dental professional would then measure the bite relationship between the patient's upper teeth and the patient's lower teeth in order to determine a comfortable mandibular advancement position for the patient. The dental professional would couple a bite gauge measurement device to the patient's upper teeth and the patient's lower teeth. The dental profession would do this by engaging a first dental fork to the patient's upper teeth and a second dental fork the patient's lower teeth. Both the first dental fork and the second dental fork would be coupled to measurement gauge on their handles. The measurement gauge and the dental forks are used to measure the distance between the patient's upper teeth and the patient's lower teeth in one direction. The patient would be instructed to protrude their lower jaw forward a distance of 3.0 mm to 5.0 mm, at which point the patient would relax and a measurement would be taken. The patient would be observed for at least 30 minutes in order to determine that the patient is not experiencing any pain or discomfort in the aforementioned position. After a determination is made that the patient is not in any pain or discomfort, an impression would be made of the patient's advanced jaw positioning for use in fusing the mandibular dental arch 2 and the maxillary dental arch 1. The impression would be using an impression material that can include but is not limited to polyvinylsiloxane as well as any polymer, copolymer, synthetic resin, and combination thereof. It should be noted that the step of measuring the bite relationship does not necessarily occur following the step of creating a set of impression from the patient's upper teeth and the patient's lower teeth and as a result can occur prior to the creation of the set of impressions.

With the set of impressions for the patient's upper teeth and the patient's lower teeth, the dental professional would create a pair of stone models. The dental professional would use the impression corresponding to the patient's upper teeth to form a maxillary stone model comprising maxillary teeth, a gingival region, and a palatal region. The dental professional would also use the impression corresponding to the patient's lower teeth to form a mandibular stone model comprising mandibular teeth, a gingival region, and a lingual region. Both the maxillary stone model and the mandibular stone model would be formed using a casting compound that would be poured into the impressions corresponding to the patient's upper teeth and the patient's lower teeth. The stone models would be dried and allowed to set leaving a hardened stone model of the patient's upper teeth and patient's lower teeth. The lower portion of the maxillary stone model corresponding, at least in part with the palatal region and the gingival region would be trimmed to a height ranging between 1.0 mm and 2.0 mm from the gingival line where the maxillary teeth meet the gingival region. The maxillary stone model is trimmed in order to provide adequate fitment in a pressure and vacuum system as well as a modular articulator system. A rounded hole is created through the palatal region of maxillary stone model, wherein the rounded hole is provided as mounting point for a suction vent in the pressure and vacuum system. The lower portion of the mandibular stone model corresponding with the lingual region and the gingival region would be trimmed to a height ranging between 1.0 mm and 2.0 mm from the gingival line where the mandibular teeth meet the gingival region. The mandibular stone model is trimmed in order to provide adequate fitment in a pressure and vacuum system as well as a modular articulator system. A rounded hole is created through the lingual region of mandibular stone model, wherein the rounded hole is provided as mounting point for a suction vent in the pressure and vacuum system. It should be noted that the rounded hole created in the palatal region of the maxillary stone model and the rounded hole created in the lingual region of the mandibular stone model are approximately 4.0 mm in diameter but that the exact measurements of the rounded holes are dependent on the specific pressure and vacuum system utilized. The pressure and vacuum system utilized includes but is not limited to Omnivac V Vacuform Adaptor, or Dentsply Tekfit System Dualform Scan, or Glidewell Erco, or Biostart System.

With the pair of stone models created, the dental professional would then create a pair of dental arches. The dental professional would place the maxillary stone model and the mandibular stone model into a pressure and vacuum system. The maxillary stone model would be aligned with a suction vent of the pressure and vacuum system, wherein the rounded hole in the palatal region of the maxillary stone model would be placed coincident with the suction vent in order to allow for greater suction. Following the installation of the maxillary stone model within the pressure and vacuum system, a polymer sheet with a thickness of at least 0.04 mm is positioned on top of the maxillary stone model. Concurrently, the mandibular stone model would be aligned with another suction vent of the pressure and vacuum system, wherein the rounded hole in the lingual region of the mandibular stone model would be placed coincident with the other suction vent in other to allow for greater suction. After the installation of the mandibular stone model within the pressure and vacuum system, another polymer sheet with a thickness of at least 0.04 mm is positioned on top of the mandibular stone model. The polymer sheet varies in thickness based on the bruxer diagnosis by the dental profession. The thickness of the polymer sheet would be determined to be a thickness of 0.04 mm is the patient was diagnosed as not being a bruxer or being a mild bruxer, 0.06 mm if the patient was diagnosed as being a medium bruxer, and 0.08 mm if the patient was diagnosed as being a severe bruxer. After the polymer sheets have been placed on top of both the maxillary stone model and the mandibular stone model, the pressure and vacuum system is activated according to the instructions of the particular pressure and vacuum system. It should be noted that the polymer sheet comprises ethylene vinyl acetate (EVA) as well as other polymer materials. The activation of the pressure and vacuum system forms the maxillary dental arch 1 and the mandibular dental arch 2. The maxillary stone model with the attached maxillary dental arch 1 and the mandibular stone model with the attached mandibular dental arch 2 are removed from the pressure and vacuum system.

With the formation of the maxillary dental arch 1 and the mandibular dental arch 2, the maxillary stone model and the mandibular stone model are removed from the pressure and vacuum system. The maxillary dental arch 1 is trimmed to 1.0 mm to 2.0 mm from the maxillary teeth on the areas corresponding to the gingival region and the palatal region. The mandibular dental arch 2 is trimmed to 1.0 mm to 2.0 mm from the mandibular teeth on the areas corresponding to the gingival region and the lingual region. Both the maxillary dental arch 1 and the mandibular dental arch 2 are separated from the maxillary stone model and the mandibular stone model, respectively. Following the separation from the maxillary stone model and the mandibular stone model, the maxillary dental arch 1 and the mandibular dental arch 2 are smoothed to reduce abrasion to the patient. The separation of the maxillary dental arch 1 and the mandibular dental arch 2 from their respective stone models occurs in order to inspect the successful formation of the maxillary dental arch 1 and the mandibular dental arch 2.

With the formation of the maxillary dental arch 1 and the mandibular dental arch 2, the dental professional would proceed to fuse the arches together forming the sleep apnea dental appliance. The maxillary stone model and the mandibular stone model would be placed into a modular articulator system. The modular articulator system is a flexible and semi-adjustable instrument that is utilized to achieve the particular mandibular advancement that was measured during the bite gauge measurement. With the maxillary stone model and the mandibular stone model engaged with the modular articulator system, the maxillary stone model and the mandibular stone model would be aligned according to the measurements taken during the bite gauge measurement and the impression created from the patient's advanced jaw positioning. The maxillary dental arch 1 would be repositioned onto the maxillary stone model while the mandibular dental arch 2 would be repositioned onto the mandibular stone model. A cold curing resin would be applied to the maxillary dental arch 1 and the mandibular dental arch 2 to the areas that coincident with a patient's premolar teeth and the patient's molar teeth. The cold curing resin would be applied to both sides of the maxillary dental arch 1 and the mandibular dental arch 2. The cold curing resin would be applied in a thickness of 3.0 mm in order to ensure a semi-rigid construction. The application of the cold curing resin would then be followed by the positioning the modular articulator system within a pressure and vacuum system in order to form the sleep apnea dental appliance. With the modular articulator system positioned within the pressure and vacuum system, the pressure and vacuum system would be activated forming the sleep apnea dental appliance. After which, the modular articulator system would be removed from the pressure and vacuum system and the sleep apnea dental appliance is removed from the modular articulator system. The cold curing resin would fuse the maxillary dental arch 1 and the mandibular dental arch 2 together forming a first advancement structure 8 and a second advancement structure 9. The sleep apnea dental appliance is then smoothed to reduce abrasion to the patient. The sleep apnea dental appliance is smoothed in the areas that are coincident with the buccal mucosa. The cold curing resin that fuses the maxillary dental arch 1 and the mandibular dental arch 2 is a pliable material that forms the first advancement structure 8 and the second advancement structure 9 that are sturdy enough to allow the sleep apnea dental appliance to withstand the torsion and compressive forces exerted by the patient's jaw. The first advancement structure 8 and the second advancement structure 9 are pliable and sturdy reducing the need for additional materials such as mental or rubber bands to join the maxillary dental arch 1 and the mandibular dental arch 2. The construction of the first advancement structure 8 and the second advancement structure 9 is smoother than existing and more comfortable than existing mandibular advancement devices resulting in increased compliance by patients. It should be noted that the pressure and vacuum system utilized in the fusion of the maxillary dental arch 1 and the mandibular dental arch 2 does not necessarily have to be the same pressure and vacuum system utilized in the formation of the maxillary dental arch 1 and the mandibular dental arch 2, and can be a pressure cooker or autoclave system.

In an additional embodiment of the present invention, the sleep apnea dental appliance can be configured for use as an oral and systemic medication system that can be applied at night or during the day. The sleep apnea dental appliance would provide anti-acidic medication at night to counter act acid wear during the day. The sleep apnea dental appliance can additionally include fluoride that would help prevent tooth decay. Furthermore the sleep apnea dental appliance could additionally include anti-dry mouth medications to help with xerostomia.

In an additional embodiment of the present invention, the sleep apnea dental appliance could be made using technology that would allow the sleep apnea dental appliance to be disposable. This could be accomplished by having a robot assist in the manufacturing of the maxillary dental arch and the mandibular dental arch. A dental professional would still be required to fuse the maxillary dental arch and the mandibular dental arch but the overall system would be more hygienic.

Although the invention has been explained in relation to its preferred embodiment, it is to be understood that many other possible modifications and variations can be made without departing from the spirit and scope of the invention as hereinafter claimed.

What is claimed is:

1. An sleep apnea dental appliance comprises:
a mandibular dental arch;
a maxillary dental arch;
a first advancement structure;
a second advancement structure;
both the mandibular dental arch and the maxillary dental arch each comprise a first molar region, a second molar region, a first premolar region, a second premolar region, and a cuspid/incisor region;
the mandibular dental arch being positioned parallel to the maxillary dental arch;
the first advancement structure and the second advancement structure being engaged between the maxillary dental arch and the mandibular dental arch;
the mandibular dental arch being offset from the maxillary dental arch.
the cuspid/incisor region being arcuately positioned between the first premolar region and the second premolar region;
the first premolar region being arcuately positioned between the cuspid/incisor region and the first molar region;
the second premolar region being arcuately positioned between the cuspid/incisor region and the second molar region;
the first molar region and the first premolar region of the mandibular dental arch being at least in part fused to the first advancement structure;
the first molar region and the second premolar region of the maxillary dental arch being at least in part fused to the first advancement structure, opposite the mandibular dental arch;
the second molar region and the second premolar region of the mandibular dental arch being at least in part fused to the second advancement structure; and
the second molar region and the second premolar region of the maxillary dental arch being at least in part fused to the second advancement structure, opposite the mandibular dental arch.

2. The sleep apnea dental appliance in claim 1 wherein the mandibular dental arch, the maxillary dental arch, the first advancement structure and the second advancement structure being constructed of a pliable material.

3. The sleep apnea dental appliance in claim 1 wherein the mandibular dental arch and the maxillary dental arch being of thickness ranging between 0.04 mm and 0.08 mm in thickness.

4. The sleep apnea dental appliance in claim 1 wherein the first advancement structure and the second advancement structure being about 3.0 mm in thickness.

5. The sleep apnea dental appliance in claim 1 wherein the cuspid/incisor region of the mandibular dental arch being horizontally offset by 3.0 mm to 5.0 mm from the cuspid/incisor region of the maxillary dental arch.

6. A method of constructing an sleep apnea dental appliance comprises the steps of:
receiving a patient diagnosed with obstructive sleep apnea;
determining the patient qualifies for the sleep apnea dental appliance;
creating a set of impressions from the patient's upper teeth and a patient's lower teeth;
measuring the bite relationship between the patient's upper teeth and the patient's lower teeth;
creating a pair of stone models from the set of impressions;
placing a maxillary stone model within a pressure and vacuum system, wherein a rounded hole through the palatal region of the maxillary stone model is aligned with a suction vent of the pressure and vacuum system;
placing a polymer sheet with a thickness of at least 0.04 mm on top of the maxillary stone model, wherein the thickness of the polymer sheet ranges between 0.04 mm to 0.08 mm in thickness;
placing a mandibular stone model within the pressure and vacuum system, wherein a rounded hole through the lingual region of the mandibular stone model is aligned with another suction vent of the pressure and vacuum system;
placing another polymer sheet with a thickness of at least 0.04 mm on top of the mandibular stone model, wherein the thickness of the other polymer sheet ranges between 0.04 mm to 0.08 mm in thickness;
activating the pressure and vacuum system forming a maxillary polymer arch and a mandibular polymer arch;
removing the maxillary stone model with the attached maxillary polymer arch and the mandibular stone model with the attached mandibular polymer arch from the pressure and vacuum system;
trimming the maxillary dental arch 1.0 mm to 2.0 mm from maxillary teeth on the gingival region and the palatal region;
trimming the mandibular dental arch 1.0 mm to 2.0 mm from mandibular teeth on the gingival region;
positioning a maxillary stone model with the attached maxillary dental arch within a modular articulator system;
positioning a mandibular stone model with the attached mandibular dental arch within the modular articulator system
aligning the maxillary stone model and the mandibular stone model within the modular articulator system based on an impression of the patient's advanced jaw positioning;
applying the cold curing resin to the maxillary dental arch and the mandibular dental arch to areas coincident with a patient's premolar teeth and a patient's molar teeth, wherein the cold curing resin is applied in a thickness of 3.0 mm;
positioning the modular articulator system within a pressure and vacuum system;
activating the pressure and vacuum system forming sleep apnea dental appliance;
removing the modular particular system from the pressure and vacuum system;
removing the sleep apnea dental appliance from the modular articulator system;
smoothing the sleep apnea dental appliance;

7. The step of creating a set of impression as claimed in claim 6 comprises the steps of:
activating a molding putty in order to create a set of impressions;
placing an amount of the molding putting into a first tray;
placing an amount of the molding putty into a second tray;
aligning the molding putty in the first tray with the patient's upper teeth;
surrounding the patient's upper teeth with the molding putty of the first tray;
aligning the molding putty in the second tray with the patient's lower teeth;
surrounding the patient's lower teeth with the molding putty of the second tray;
removing the molding putty of the first tray from the patient's upper teeth after a particular amount of time, wherein the molding putty of the first tray has the impressions of the patient's upper teeth; and
removing the molding putty of the second tray from the patient's lower teeth after a particular amount of time, wherein the molding putty of the second tray has the impressions of the patient's lower teeth.

8. The step of measuring the bite relationship as claimed in claim 6 comprises the steps of:
coupling a bite gauge measurement device to a patient's upper and lower teeth;
instructing patient to protrude their lower jaw forward, wherein the patient is asked to move their lower jaw a distance of 3.0 mm to 5.0 mm forward;
measuring the forward positioning of the patient's lower jaw;
observing the patient for of at least 30 minutes, wherein the patient is observed in order to ensure that no pain or discomfort is felt in the advanced position; and
creating an impression of the patient's advanced jaw position.

9. The step of creating a pair of stone models as claimed in claim 6 comprises the steps of:
creating a maxillary stone model from an impression of the patient's upper teeth of the set of impressions, wherein the maxillary stone model comprises maxillary teeth, gingival region, and a palatal region;
creating a mandibular stone model from an impression of the patient's lower teeth of the set of impressions, wherein the mandibular stone model comprises mandibular teeth, a gingival region, and a lingual region;
trimming the maxillary stone model to a height of 1.0 mm to 2.0 mm from the maxillary teeth on the gingival region;
trimming the mandibular stone model to a height of a 1.0 mm to 2.0 mm from the mandibular teeth on the gingival region;
creating a rounded hole through the palatal region of the maxillary stone model; and
creating a rounded hole through the lingual region of the mandibular stone model.

10. A method of constructing an sleep apnea dental appliance comprises the steps of:
receiving a patient diagnosed with obstructive sleep apnea;
determining the patient qualifies for the sleep apnea dental appliance;
activating a molding putty in order to create a set of impressions;
placing an amount of the molding putting into a first tray;
placing an amount of the molding putty into a second tray;
aligning the molding putty in the first tray with the patient's upper teeth;
surrounding the patient's upper teeth with the molding putty of the first tray;
aligning the molding putty in the second tray with the patient's lower teeth;
surrounding the patient's lower teeth with the molding putty of the second tray;
removing the molding putty of the first tray from the patient's upper teeth after a particular amount of time, wherein the molding putty of the first tray has the impressions of the patient's upper teeth;
removing the molding putty of the second tray from the patient's lower teeth after a particular amount of time, wherein the molding putty of the second tray has the impressions of the patient's lower teeth;
coupling a bite gauge measurement device to a patient's upper and lower teeth;
instructing patient to protrude their lower jaw forward, wherein the patient is asked to move their lower jaw a distance of 3.0 mm to 5.0 mm forward;
measuring the forward positioning of the patient's lower jaw;
observing the patient for of at least 30 minutes, wherein the patient is observed in order to ensure that no pain or discomfort is felt in the advanced position;
creating an impression of the patient's advanced jaw position;
creating a maxillary stone model from an impression of the patient's upper teeth of the set of impressions, wherein the maxillary stone model comprises maxillary teeth, gingival region, and a palatal region;
creating a mandibular stone model from an impression of the patient's lower teeth of the set of impressions, wherein the mandibular stone model comprises mandibular teeth, a gingival region, and a lingual region;
trimming the maxillary stone model to a height of 1.0 mm to 2.0 mm from the maxillary teeth on the gingival region;
trimming the mandibular stone model to a height of a 1.0 mm to 2.0 mm from the mandibular teeth on the gingival region;
creating a rounded hole through the palatal region of the maxillary stone model;
creating a rounded hole through the lingual region of the mandibular stone model;
placing a maxillary stone model within a pressure and vacuum system, wherein a rounded hole through the palatal region of the maxillary stone model is aligned with a suction vent of the pressure and vacuum system;
placing a polymer sheet with a thickness of at least 0.04 mm on top of the maxillary stone model, wherein the thickness of the polymer sheet ranges between 0.04 mm to 0.08 mm in thickness;
placing a mandibular stone model within the pressure and vacuum system, wherein a rounded hole through the lingual region of the mandibular stone model is aligned with another suction vent of the pressure and vacuum system;
placing another polymer sheet with a thickness of at least 0.04 mm on top of the mandibular stone model, wherein the thickness of the other polymer sheet ranges between 0.04 mm to 0.08 mm in thickness;
activating the pressure and vacuum system forming a maxillary polymer arch and a mandibular polymer arch;
removing the maxillary stone model with the attached maxillary polymer arch and the mandibular stone model with the attached mandibular polymer arch from the pressure and vacuum system;
trimming the maxillary dental arch 1.0 mm to 2.0 mm from maxillary teeth on the gingival region and the palatal region;
trimming the mandibular dental arch 1.0 mm to 2.0 mm from mandibular teeth on the gingival region;
positioning a maxillary stone model with the attached maxillary dental arch within a modular articulator system;
positioning a mandibular stone model with the attached mandibular dental arch within the modular articulator system
aligning the maxillary stone model and the mandibular stone model within the modular articulator system based on an impression of the patient's advanced jaw positioning;
applying the cold curing resin to the maxillary dental arch and the mandibular dental arch to areas coincident with a patient's premolar teeth and a patient's molar teeth, wherein the cold curing resin is applied in a thickness of 3.0 mm;
positioning the modular articulator system within a pressure and vacuum system;
activating the pressure and vacuum system forming sleep apnea dental appliance;
removing the modular particular system from the pressure and vacuum system;
removing the sleep apnea dental appliance from the modular articulator system; and
smoothing the sleep apnea dental appliance.
